(19) United States
(12) Patent Application Publication
(10) Pub. No.: US 2017/0071530 A1
(43) Pub. Date: Mar. 16, 2017

(54) THERMAL TOOTH TESTING DEVICES, METHODS AND KITS

(71) Applicant: Gerald KABAN, Vacaville, CA (US)

(72) Inventor: Gerald KABAN, Vacaville, CA (US)

(21) Appl. No.: 15/258,523

(22) Filed: Sep. 7, 2016

Related U.S. Application Data
(60) Provisional application No. 62/218,149, filed on Sep. 14, 2015.

(51) Int. Cl. A61B 5/00; A61C 19/04

(52) U.S. Cl. CPC A61B 5/483; A61B 5/4547; A61C 19/04

(57) ABSTRACT

The disclosed devices relate to disposable self-loading absorbent-tipped thermal tooth testing instruments. The instrument utilizes the expansion of dental refrigerant to saturate a cotton-tipped applicator which is permanently attached to one end of the tester into which the opposite end conveniently fits into a spray nozzle of any currently commercially available dental refrigerant can.

### CROSS-REFERENCE

This application claims the benefit of U.S. Provisional Application No. 62/218,149, filed Sep. 14, 2015, entitled “Thermal Tooth Testing Devices, Methods and Kits” which application is incorporated herein by reference.

### BACKGROUND

The present disclosure relates to a disposable self-loading absorbable-tipped cold thermal tooth testing instrument.

In dentistry teeth are routinely thermally cold tested to gauge a patient's response. This process typically involves a cotton pliers/forceps or foam pellet that has been sprayed with a commercially available dental refrigerant. This cotton pliers/forceps assembly is then placed directly on the surface of a dental patient's tooth and the patient's response is noted. The purpose of the test is to determine the response of the nerve within a patient's tooth to cold which provides diagnostic information as to the condition of the nerve. This test is also utilized to assess the patient's response to local anesthetic during dental procedures. The disadvantage of this current method is it involves multiple steps, multiple items and two hands or two individuals to properly execute and because the refrigerant is applied by directly spraying the outside of the cotton pellet or foam applicator the time and amount of applied refrigerant varies which leads to inaccurate thermal testing results.

### SUMMARY

Disclosed is a disposable device with an elongated member having an absorbable tip which can be loaded with refrigerant prior to use.

An aspect of the disclosure is directed to thermal testing instruments. Suitable instruments comprise: an elongated tubular member having a proximal end and a distal end wherein the elongated tubular member has an exterior tubular member and an interior tubular member positioned within an interior of the exterior tubular member; an applicator tip positioned at a distal end of the elongated tubular member wherein the applicator tip is in fluid communication with an interior of the interior tubular member. Additionally, the elongated tubular member can be configurable to be bendable along its length, either during the manufacturing process or by the user prior to use. A removable envelop can be provided which is positioned about the applicator tip. Additionally, the applicator tip can be formed from any suitable absorbent material, including cotton or foam. The elongated tubular member can be formed of a suitable plastic. Additionally, the interior tubular member can have a cross-sectional that defines a spacing between an inner surface of the exterior tubular member and an exterior surface of the interior tubular member. Moreover, the cross-sectional shape of the interior tubular member is selected from star, square, and ovoid. The cross-sectional shape of the interior tubular member is non-circular.

Another aspect of the disclosure is directed to a means for thermal testing. Suitable means comprise: an elongated tubular member means having a proximal end and a distal end wherein the elongated tubular member means has an exterior tubular member means and an interior tubular member means positioned within an interior of the exterior tubular member; an applicator tip means positioned at a distal end of the elongated tubular member means wherein the applicator tip means is in fluid communication with an interior of the interior tubular member. Additionally, the elongated tubular member means is bendable along its length. In some configurations, a removable envelope means positioned about the applicator tip. The applicator tip means is cotton or foam. Additionally, the elongated tubular member means is plastic. The interior tubular member means is configurable to have a cross-sectional that defines a spacing between an inner surface of the exterior tubular member means and an exterior surface of the interior tubular member. Additionally, the cross-sectional shape of the interior tubular member means can be selected from star, square, and ovoid. Alternatively, the cross-sectional shape of the interior tubular member means can be any non-circular shape that creates a space between the interior tubular member means and the interior surface of the exterior tubular member means.

Still another aspect of the disclosure is directed to methods of thermal testing a tooth. Suitable methods comprise: engaging a refrigerant can with a thermal testing instrument having an elongated tubular member has an exterior tubular member and an interior tubular member positioned within an interior of the exterior tubular member, an applicator tip positioned at a distal end of the elongated tubular member wherein the applicator tip is in fluid communication with an interior of the interior tubular member, and a removable seal, pressing a nozzle of the refrigerant can to deliver refrigerant through the interior tubular member to the applicator tip; removing the seal from the applicator tip. Additionally, methods can include applying the applicator tip to a tooth of a patient. In some methods the elongated tubular member is bent by the user during deployment of the device.

Yet another aspect of the disclosure is directed to method of thermal testing a tooth comprising: engaging a refrigerant means with a thermal testing instrument having an elongated tubular member means has an exterior tubular member means and an interior tubular member means positioned within an interior of the exterior tubular member, an applicator tip means positioned at a distal end of the elongated tubular member means wherein the applicator tip means is in fluid communication with an interior of the interior tubular member, and a removable seal, pressing a nozzle of the refrigerant can to deliver refrigerant through the interior tubular member means to the applicator tip; removing the seal from the applicator tip. Methods can also include applying the applicator tip means to a tooth of a patient. Additionally, the user can bend the elongated tubular member means during deployment.

Another aspect of the disclosure is directed to kits for thermal testing a tooth. Suitable kits comprise: two or more thermal testing instruments comprising an elongated tubular member having a proximal end and a distal end wherein the elongated tubular member has an exterior tubular member and an interior tubular member positioned within an interior of the exterior tubular member, an applicator tip positioned at a distal end of the elongated tubular member wherein the applicator tip is in fluid communication with an interior of the interior tubular member; and a can of refrigerant.

Yet another aspect of the disclosure is directed to kits for thermal testing a tooth. Suitable kits comprise: two or more thermal testing instruments comprising an elongated tubular member means having a proximal end and a distal end wherein the elongated tubular member means has an exterior tubular member means and an interior tubular member means positioned within an interior of the exterior tubular member, an applicator tip means positioned at a distal end of the elongated tubular member means wherein the applicator tip means is in fluid communication with an interior of the interior tubular member; and a refrigerant means.

### INCORPORATION BY REFERENCE

All publications, patents, and patent applications mentioned in this specification are herein incorporated by reference to the same extent as if each individual publication, patent, or patent application was specifically and individually indicated to be incorporated by reference. See, for example, U.S. Pat. No. 2,294,186 A published Jul. 17, 1942 to Kirschbaum for Surgical Sponge and Handle; U.S. Pat. No. 4,991,750 A issued Feb. 12, 1991, to Moral for Mouting for Extension Tube; U.S. Pat. No. 5,647,746 A published Jul. 15, 1997 to Chipman for Dental Compound Applicator; U.S. Pat. No. 5,938,438 A published Aug. 19, 1999 to Chipman for Dental Compound Applicator; U.S. Pat. No. 6,592,280 published Jul. 15, 2003 to Petrich et al. for Container and Applicator Assembly; U.S. Pat. No. 8,839,994 B2 issued Sep. 23, 2014 to Mason for Aerosol Can Spray Nozzle Extension Tube Adapter; US 2002/0106609 A1 published Aug. 8, 2002 to Palermo for Dental Forceps; and US 2012/0003605 A1 published Jan. 5, 2012 to Johnsen et al. for Endodontic Diagnostic Instrument.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a side view of a thermal testing instrument according to the disclosure;

FIG. 2 is a side view of a thermal testing instrument according to the disclosure illustrating a flexible joint;

FIG. 3 is a close-up of a distal tip of a thermal testing instrument according to the disclosure;

FIG. 4 is a cross-sectional view of a thermal testing instrument along the lines 4-4 in FIG. 1;

FIG. 5 illustrates a thermal testing instrument ready for connection to a can containing a dental refrigerant according to the disclosure;

FIG. 6 illustrates the thermal testing instrument attached to a can containing a dental refrigerant;

FIG. 7 illustrates the thermal testing instrument being pre-loaded with the dental refrigerant;

FIG. 8 illustrates the thermal testing instrument as the end packaging is removed prior to testing; and

FIG. 9 illustrates the thermal testing instrument as the refrigerant cotton saturated tip is applied to a patient's tooth surface for the purpose of testing its cold thermal response.

### DETAILED DESCRIPTION

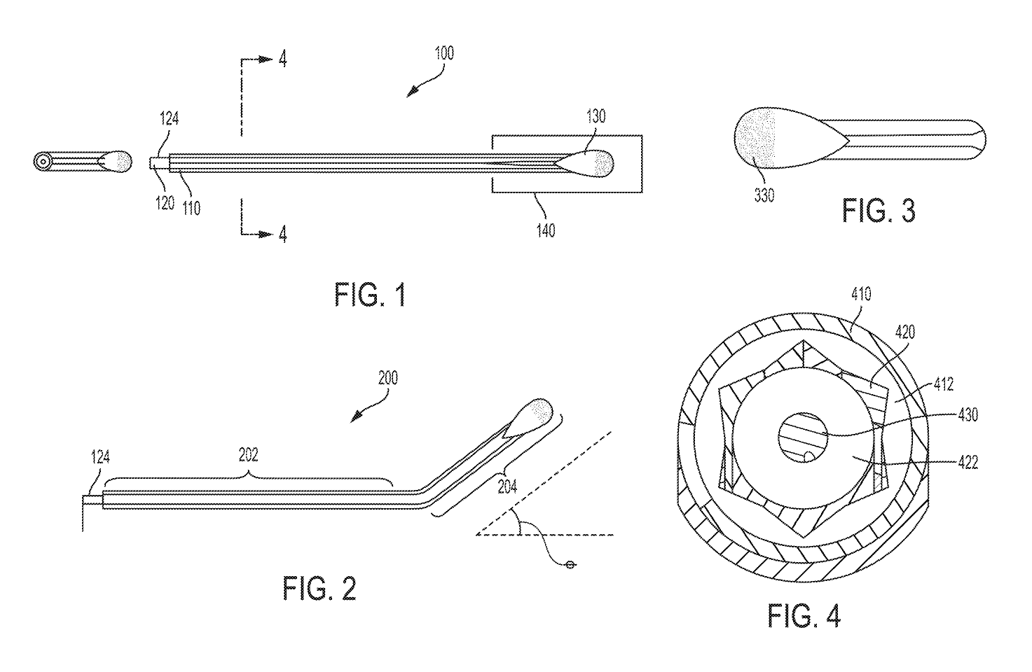

FIG. 1 illustrates a thermal testing instrument 100 having an outer elongated tubular member 110 and an inner tubular member 120 where each of the outer tubular member and inner tubular member have a wall thickness for the tubular member and a length. An applicator tip 130 is positioned at a distal end. The proximal end is configurable to engage a can containing a dental refrigerant. A removable seal 140 that wraps around the distal end of the thermal testing instrument 100 is provided around the applicator tip 130 and is in place prior to use. The removable seal 140 encapsulates the applicator tip 130 to provide a covering. The overall length of the device can be from 80 mm to 150 mm, more preferably 100 mm to 120 mm. As will be appreciated by those skilled in the art, the thermal testing instrument 100 can be comprised of a single elongated member which wherein the wall thickness of the tubular member is thicker than the wall thickness for either of the outer elongated tubular member and the inner tubular member. Additionally, the single elongated member can have a first wall thickness at a proximal end and a second wall thickness at a distal end. Providing either a thicker elongated member or a dual thickness elongated member provides insulation to the user's hands when the refrigerant passes through the thermal testing instrument. The dual tube configuration illustrated provides an air gap between the refrigerant in the inner tubular member and the user's hands engaging the outer tubular member which acts to insulate the user's hands from the temperature of the refrigerant. For the configuration using a single tubular member and a thicker wall thickness, the proximal end of the device can be milled to facilitate engaging an aerosol can with a nipple fit.

As shown in FIG. 2, the thermal testing instrument 200 has a first length 202 and a second length 204. The length of the first length 202 can be from 70 mm to 100 mm, more preferably 80 mm to 90 mm, and even more preferably 87 mm. The length of the second length 204 can be from 18 mm to 30 mm, more preferably from 20 mm to 25 mm, and even more preferably 24 mm. The thermal testing instrument 200 can bend at least at one location along its length so that an angle between an axis formed by the first length 202 and an axis formed by a second length 204 is from 20° to 45°, more preferably from 35° to 40°, and even more preferably 38°. The bend of the thermal testing instrument can be achieved during the manufacturing process, or in some configurations by the user. Typically the bend is similar to the bend of a dental mirror to facilitate ease of use and ease of placement.

The applicator tip 330 on the distal end is shown in FIG. 3. The applicator tip 330 can be formed from a No. 2 Cotton Pellet with a diameter of 7/23″ and a length of 5.5 mm. Suitable cotton pellets include cotton pellets available from Richmond Premium Dental and Medical Products, Charlotte, N.C. FIG. 4 is a cross-sectional view of the thermal testing instrument 100 shown in FIG. 1 taken along the lines 4-4 illustrating the double walled construction. An outer tubular member 410 houses an inner tubular member 420 and a central post 430. A first interior aperture 412 forms a space between the outer tubular member 410 and the inner tubular member 420. A second interior aperture 422 forms a space between the inner tubular member 420 and the central post 430. Refrigerant passes from the can through the second interior aperture 422 to the applicator tip 130 to saturate the applicator tip 130 with refrigerant. The inner tubular member 420 has a cross-sectional profile that creates a space between the outer surface of the inner tubular member 420 and the inner surface of the outer tubular member 410. Suitable cross-sectional shapes are non-circular, ovoid, square; star, etc. The configuration allows for less than 50% of the exterior surface of the inner tubular member 420 to contact the interior surface of the outer tubular member 410. More preferably, less than 30% of the surface area contact, even more preferably, less than 20% of the surface area contact.

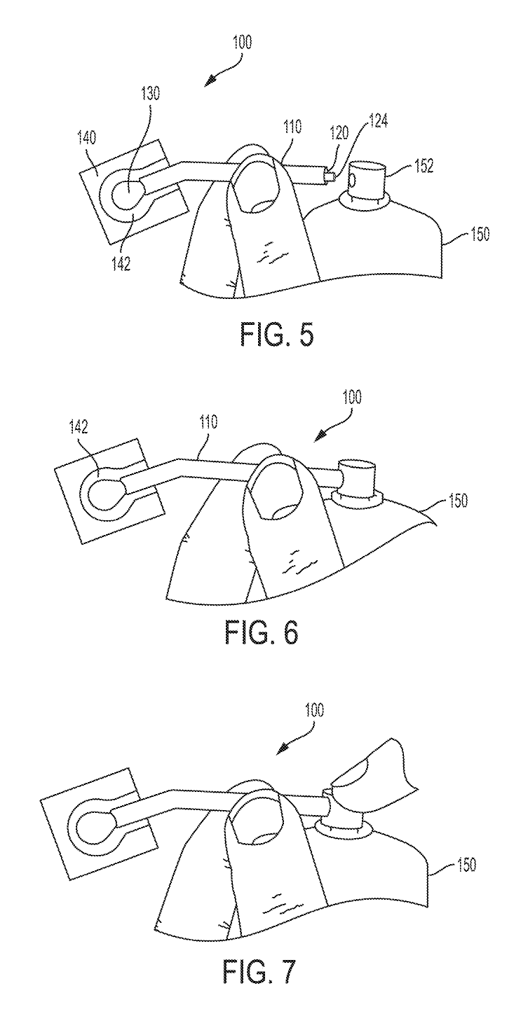

FIG. 5 is a detailed drawing of the thermal testing instrument 100 having an applicator tip 130 showing the applicator tip 130 on one end of the outer elongated tubular member 110, and the built-in plastic nipple 124 which allows attachment of the instrument to any commercially available dental refrigerant can 150. Suitable dental refrigerant includes, for example, Endo-Ice® Pulp Vitality Refrigerant Spray, manufactured by Coltene/Whaledent Inc. (Cuyahoga Falls, Ohio) or ENDO Freeze™ manufactured by Select Dental Manufacturing, (Farmingdale, N.Y.). The outer elongated tubular member 110 which forms part of the elongated connector can be configured with a bend along its length as described above. Also illustrated is the packaging 140 configured to house the applicator tip 130 wherein the removable seal 140 packaging is configured to create an envelope or enclosure 142 into which the refrigerant is delivered from the can 150 to totally saturate the applicator tip 130. The applicator tip 130 can, for example, be formed from cotton or foam. As depicted in FIG. 5 the thermal testing instrument 100 is about to engage the nozzle of, for example, a commercial dental refrigerant can.

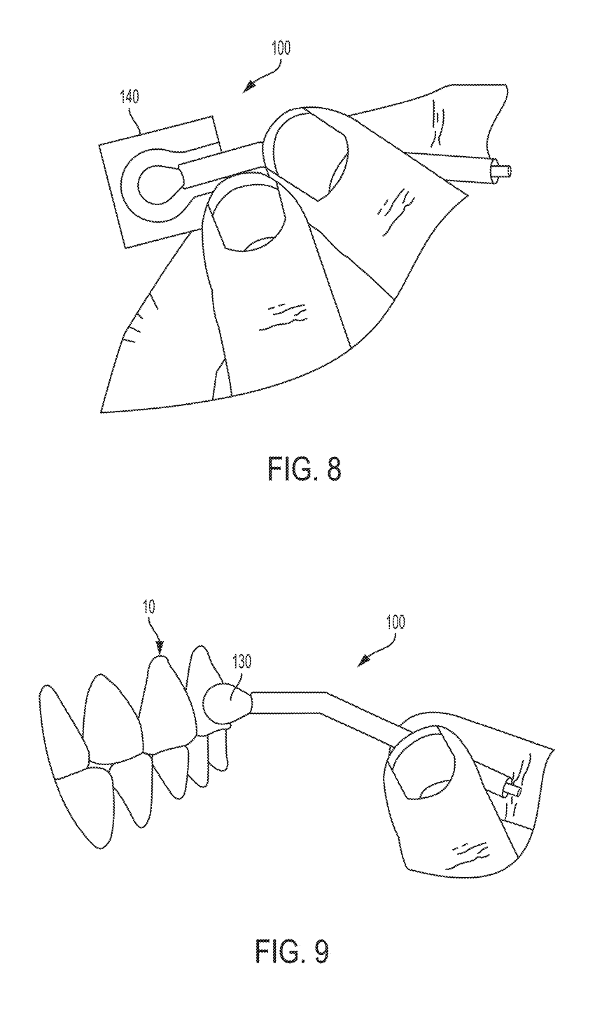

FIG. 6 is an illustration showing the thermal testing instrument 100 attached to the dental refrigerant can 150. FIG. 7 illustrates the thermal testing instrument 100 being pre-loaded (i.e., loading before use) with the dental refrigerant from the refrigerant can 150. FIG. 8 illustrates the thermal testing instrument 100 as the user is preparing to remove the end packaging 110 prior to testing a patient's teeth for sensitivity. FIG. 9 is an illustration of the thermal testing instrument 100 after removal of the packaging 110 (shown in FIG. 8) and following saturation of the applicator as the refrigerant saturated applicator tip is applied to a patient's tooth surface 10 for the purpose of testing the patient's cold thermal response.

In use, the disposable self-loading cotton-tipped thermal tooth testing instrument provides for an expansion of dental refrigerant to saturate an applicator tip 130 which is attached to one end of the thermal testing instrument 100 into which the proximal end of the thermal testing instrument 100 fits into a spray nozzle of a commercially available dental refrigerant can. The refrigerant nozzle 152 is depressed by the user allowing the refrigerant within the refrigerant can 150 to travel through the double-walled, hollow tube forming the tubular member of the thermal testing instrument 100 to saturate the applicator tip 130 at the opposite, distal end. Another feature is the innovative individual packaging of the distal end of each instrument with a removable seal 140 optimizes saturation of the applicator tip 130 within the enclosure 142 of the removable seal 140 before removal of the applicator tip 130 from the removable seal 140. Once saturated, the thermal testing device is detached from the refrigerant can 150, the removable seal 140 comprising the individual packing is then removed and the thermal testing instrument 100 is carried directly to the patient's tooth to conduct the dental cold (thermal) test. The disposable self-loading cotton-tipped thermal tooth tester can have a bend close to the cotton tip as described above which facilitates easier placement of the refrigerant saturated applicator tip 130 on the patient's tooth for performing the cold (thermal) testing. At the termination of the thermal test the instrument is discarded.

The double-walled hollow plastic tube shown in FIG. 4 allows the refrigerant to travel from the refrigerant source (refrigerant can 150) to saturate the applicator tip. The double walled configuration insulates the outer tube which is also the hand-held portion of the thermal testing instrument 100. The proximal end of the thermal testing instrument 100, opposite the applicator tip 130, is designed with an extension of the inner tube which allows attachment of the instrument to any commercially available dental refrigerant can. This instrument allows for self-loading of the refrigerant directly to the cotton tip and is a departure from the current methods that directly spray refrigerant on a pellet or foam device to then continue with the cold (thermal) test.

In use, the dentist or assistant, connects the thermal testing instrument to a refrigerant can nozzle. The nozzle is depressed to cause refrigerant from within the can to exit the can through the nozzle and through an inner lumen of the double walled elongated member to the applicator tip positioned within the packaging. Once a desired amount of refrigerant has been applied to the applicator, the dentist disengages the device from the nozzle and the device is ready for use on a patient. The packaging is removed, and the applicator tip is then applied to one or more teeth of the patient. Thereafter, the applicator is thrown away.

As will be appreciated by those skilled in the art, the devices disclosed herein can be compiled into kits where a plurality of disposable devices are placed within suitable packaging. Additionally, a plurality of devices can be connected to one another such that the user removes one device at a time from a length comprising a plurality of devices. Kits can include one or more thermal testing instruments and a can of refrigerant.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A thermal testing instrument comprising:
an elongated tubular member having a proximal end and a distal end wherein the elongated tubular member has an exterior tubular member and an interior tubular member positioned within an interior of the exterior tubular member;
an applicator tip positioned at a distal end of the elongated tubular member wherein the applicator tip is in fluid communication with an interior of the interior tubular member.

2. The thermal testing instrument of claim 1 wherein the elongated tubular member is bendable along its length.

3. The thermal testing instrument of claim 1 further comprising a removable envelop positioned about the applicator tip.

4. The thermal testing instrument of claim 1 wherein the applicator tip is cotton or foam.

5. The thermal testing instrument of claim 1 wherein the elongated tubular member is plastic.

6. The thermal testing instrument of claim 1 wherein the interior tubular member has a cross-sectional that defines a spacing between an inner surface of the exterior tubular member and an exterior surface of the interior tubular member.

7. The thermal testing instrument of claim 6 wherein the cross-sectional shape of the interior tubular member is selected from star, square, and ovoid.

8. The thermal testing instrument of claim 6 wherein the cross-sectional shape of the interior tubular member is non-circular.

9. A method of thermal testing a tooth comprising:
engaging a refrigerant can with a thermal testing instrument having an elongated tubular member has an exterior tubular member and an interior tubular member positioned within an interior of the exterior tubular member, an applicator tip positioned at a distal end of the elongated tubular member wherein the applicator tip is in fluid communication with an interior of the interior tubular member, and a removable seal,
pressing a nozzle of the refrigerant can to deliver refrigerant through the interior tubular member to the applicator tip;
removing the seal from the applicator tip.

10. The method of thermal testing of claim 9 further comprising, applying the applicator tip to a tooth of a patient.

11. The method of thermal testing of claim 9 further comprising, bending the elongated tubular member.

12. A kit comprising:
two or more thermal testing instruments comprising an elongated tubular member having a proximal end and a distal end wherein the elongated tubular member has an exterior tubular member and an interior tubular member positioned within an interior of the exterior tubular member, an applicator tip positioned at a distal end of the elongated tubular member wherein the applicator tip is in fluid communication with an interior of the interior tubular member; and
a can of refrigerant.

13. A means for thermal testing comprising:
an elongated tubular member means having a proximal end and a distal end wherein the elongated tubular member means has an exterior tubular member means and an interior tubular member means positioned within an interior of the exterior tubular member;
an applicator tip means positioned at a distal end of the elongated tubular member means wherein the applicator tip means is in fluid communication with an interior of the interior tubular member.

14. The means for thermal testing of claim 13 wherein the elongated tubular member means is bendable along its length.

15. The means for thermal testing of claim 13 further comprising a removable envelope means positioned about the applicator tip.

16. The means for thermal testing of claim 13 wherein the applicator tip means is cotton or foam.

17. The means for thermal testing of claim 13 wherein the elongated tubular member means is plastic.

18. The means for thermal testing of claim 13 wherein the interior tubular member means has a cross-sectional that defines a spacing between an inner surface of the exterior tubular member means and an exterior surface of the interior tubular member.

19. The means for thermal testing of claim 18 wherein the cross-sectional shape of the interior tubular member means is selected from star, square, and ovoid.

20. The means for thermal testing of claim 18 wherein the cross-sectional shape of the interior tubular member means is non-circular.

21. A method of thermal testing a tooth comprising:
engaging a refrigerant means with a thermal testing instrument having an elongated tubular member means has an exterior tubular member means and an interior tubular member means positioned within an interior of the exterior tubular member, an applicator tip means positioned at a distal end of the elongated tubular member means wherein the applicator tip means is in fluid communication with an interior of the interior tubular member, and a removable seal,
pressing a nozzle of the refrigerant can to deliver refrigerant through the interior tubular member means to the applicator tip;
removing the seal from the applicator tip.

22. The method of thermal testing of claim 21 further comprising, applying the applicator tip means to a tooth of a patient.

23. The method of thermal testing of claim 21 further comprising, bending the elongated tubular member means.

24. A kit comprising:
two or more thermal testing instruments comprising an elongated tubular member means having a proximal end and a distal end wherein the elongated tubular member means has an exterior tubular member means and an interior tubular member means positioned within an interior of the exterior tubular member, an applicator tip means positioned at a distal end of the elongated tubular member means wherein the applicator tip means is in fluid communication with an interior of the interior tubular member; and
a refrigerant means.
